(19) United States
(12) Patent Application Publication
(10) Pub. No.: US 2026/0369428 A1
(43) Pub. Date: Nov. 19, 2026

(54) SYSTEM FOR MANAGED COMMUNICATION BETWEEN PATIENTS AND HEALTHCARE PROVIDERS

(71) Applicant: The Texas A&M University System, College Station, TX (US)

(72) Inventors: Nancy R. Downing, College Station, TX (US); Farzan Sasangohar, Houston, TX (US)

(21) Appl. No.: 19/678,959

(22) Filed: May 15, 2026

Related U.S. Application Data
(60) Provisional application No. 63/806,445, filed on May 15, 2025.

(51) Int. Cl. G16H 80/00; H04L 67/12

(52) U.S. Cl. CPC G16H 80/00; H04L 67/12

(57) ABSTRACT

Systems and methods for facilitating communications between patients and healthcare providers may route messages and other communications through a backend service that replaces contact identifiers in inbound messages with abstract patient identifiers to prevent exposure of patient contact information to providers, and stores inbound and outbound messages and/or other communications in a database for later retrieval and review. Healthcare providers may view inbound messages and provide input for response, including reply messages or requests to initiate calls, via a user interface. Alternatively or additionally, outbound messages may be automatically generated based on message logic and automated analysis of inbound message contents.

### CLAIM OF PRIORITY

This patent application claims the benefit of priority to U.S. Provisional Application Ser. No. 63/806,445, filed May 15, 2025, which is incorporated by reference herein in its entirety.

### BACKGROUND

In many healthcare scenarios, health outcomes can be improved by maintaining communication between patients and healthcare providers beyond hospitalizations, clinic visits, or office visits. For example, upon discharge from an emergency room or hospital, e.g., following a surgery or other treatment, it may be beneficial to provide the patient with a seamless way to obtain prescription refills or adjusted prescriptions for pain management or other medications, schedule follow-up visits, or receive answers to questions about post-treatment care. In some contexts, such as maternity and neonatal care, where questions and new problems may frequently arise, a communication channel with healthcare providers can provide timely answers and practical advice, as well as to direct the patient to further resources as needed. Further, patients who have experienced some kind of trauma, such as cancer, a severe injury, or sexual assault, domestic violence, or child abuse, may benefit from regular mental health checks and quick access to mental health care.

### SUMMARY

The present disclosure relates to systems and methods for facilitating structured and controlled communication between patients and healthcare providers, for example, in contexts where ongoing engagement, follow-up, and documentation are important. In various embodiments, the system includes a backend service that functions as a central gateway for routing and managing bidirectional message flow, a provider-facing user interface that aggregates inbound messages across patients, and a database in which inbound and outbound communications are persistently recorded for later review. Collectively, the backend service, user interface, and database operate to mediate auditable communication between patients and healthcare providers. The system may implement a hybrid communication model that combines automated and provider-driven communications, for example, by utilizing automated outbound messages for rapid responses or scheduled or rule-based follow-up, while enabling provider interaction with patients via the user interface. Further, the system may support escalation from asynchronous messaging to synchronous communication modalities, such as phone calls or video sessions. In some embodiments, the backend service further performs automated analysis of the content of inbound messages, for example, to determine attributes such as urgency or categorization. The results of such analysis may influence both the generation or selection of content for automated responses and the presentation of inbound messages within the user interface, where the messages may be visually displayed in a manner that reflects, for instance, relative prioritization and/or categories, including via indicators corresponding to the inbound messages that reflect such attributes.

In various embodiments, the system is configured for alias-based internal message routing using abstract patient identifiers in place of patients'contact identifiers, such as phone numbers or email addresses. This abstraction layer enables patient-provider communication without exposing patient contact information to healthcare providers. The backend service, upon receipt of an inbound message from the patient, may replace the contact identifier associated with the inbound message with an abstract patient identifier assigned to the patient before storing the inbound message in the database and/or sending the inbound message or notification thereof to the user interface. Conversely, upon receipt of provider input for an outbound communication to a patient identified by the abstract patient identifier, the backend service may resolve the abstract patient identifier to the corresponding contact identifier when initiating the outbound communication. By preventing exposure of contact identifiers, the system enhances patient privacy and reduces the likelihood of unauthorized or accidental disclosure of patient contact information. Moreover, backend-mediated, alias-based routing enforces a controlled communication channel through which all inbound messages, provider-generated and automatically generated outbound messages, and calls flow, enabling consistent tracking, auditing, and application of communication policies. Centralizing identifier resolution and communication logic within the backend service also enables consistent enforcement of workflow rules across communication modalities, regardless of specific endpoints or channels used.

In some embodiments, the system integrates an (optional) intervening messaging service that interfaces between the backend service and external communication networks. The backend service may communicate with the messaging service via standard application programming interfaces (APIs) to receive inbound messages, deliver outbound messages, or initiate calls or other live communication sessions. Such an arrangement enables the system to be deployed with minimal integration into existing messaging or telephony infrastructure, as the messaging service need not be modified to support abstract identifiers or backend-specific logic. Alternatively, the functionality of the messaging service may be integrated into the backend service itself.

The backend service may include a message analysis component and message logic configured to process inbound messages and determine corresponding system actions. The message analysis component may analyze the content of inbound messages, for example, using keyword detection, rule-based processing, or machine-learning models, to determine one or more attributes associated with each inbound message, such as urgency, category, or status. These attributes may be used to influence both the presentation of inbound messages within the user interface and the behavior of automated messaging workflows. In particular, indicators corresponding to inbound messages and presented in the user interface may reflect attributes determined from the analysis of inbound messages, including ordering, grouping, or visual characteristics that indicate urgency or category. The message logic may further use inbound messages and/or attributes determined from analysis of inbound messages to generate or select content for outbound communications. In some embodiments, outbound communications may be generated based on predefined rules, templates, or schedules, while in other embodiments, more flexible processing may be applied to tailor communications to patient context. The message logic may also initiate notifications to the user interface and support escalation workflows, including generating alerts or transitioning communications to synchronous modalities where appropriate.

The disclosed architecture may be applied in a variety of contexts involving structured, privacy-sensitive communication workflows. In some embodiments, the system may support communication with crime victims by providing secure, controlled outreach channels that preserve anonymity and facilitate follow-up care, including communications aligned with anticipated needs following clinical events. In other embodiments, the system may support programs for pregnant or postnatal women, including scheduled messaging and provider interaction aligned with anticipated care needs over time. The system may also be applied in mental health contexts, where ongoing communication and monitoring are supported by analysis of inbound messages and escalation features, or in post-surgical care, where follow-up messaging and patient-reported information may be used to monitor recovery and trigger interventions. In certain implementations, outbound communications may be timed, selected, or generated based on patient engagement, clinical timelines, or structured input from patients and subject-matter experts, thereby enabling context-sensitive, patient-centered communication workflows within a controlled and privacy-preserving system.

### BRIEF DESCRIPTION OF THE DRAWINGS

The foregoing will be more readily understood from the following detailed description of illustrative embodiments, in particular, when taken in conjunction with the accompanying drawings.

FIG. 1 is a block diagram illustrating an example computer-implemented system for managing communication between patients and healthcare providers, in accordance with various embodiments.

FIG. 2 is a flowchart illustrating an example backend-mediated workflow for facilitating communication between a patient and a healthcare provider, in accordance with various embodiments.

FIG. 3 is a flowchart illustrating an example bifurcated workflow for facilitating both asynchronous and synchronous communications between a patient and a healthcare provider, in accordance with various embodiments.

FIG. 4 is a flowchart illustrating an example workflow for analyzing inbound messages to generate user-interface indicators and alerts, in accordance with various embodiments.

FIG. 5 is a flowchart illustrating an example workflow for automatically generating outbound messages to patients, in accordance with various embodiments.

FIG. 6 is a block diagram of an example machine as may be used for implementing any one or more of the various components (e.g., backend, user interface, database) of the system of FIG. 1 and the workflows of FIGS. 2-5.

### DESCRIPTION

Disclosed herein are computer-implemented systems and methods for establishing and maintaining lines of communication between patients and healthcare providers, using an architecture that preserves patient privacy while enabling centralization of communications across patients and healthcare providers and supporting record-keeping of such communications. Various embodiments integrate workflows for generating automated messages to patients, and/or for organizing and/or prioritizing messages received from patients for presentation to healthcare providers.

FIG. 1 is a block diagram illustrating an example computer-implemented system 100for managing communication between patients and healthcare providers, in accordance with various embodiments. The system 100 is implemented by one or more networked computing devices collectively configured to exchange communications with one or more patient devices 102 over a suitable communications network, such as the Internet or another wide-area or local-area network. The patient devices 102 may include, for example, a mobile phone, tablet, laptop, desktop computer, or other computing device capable of sending and receiving messages (e.g., text messages or multimedia messages) as well as, in many cases, of placing or receiving calls or establishing other live communication channels (such as voice-over-IP calls or video sessions).

In some embodiments, as shown in FIG. 1, the system 100 interfaces with the one or more patient devices 102 via an intermediate messaging service 104. The messaging service 104may include, for example, a short message service (SMS) gateway, a multimedia messaging service (MMS) gateway, a telephony service provider, or a communication platform that provides application programming interfaces (APIs) for programmatic sending and receiving of communications, thereby enabling the system 100 to send and receive external communications without requiring direct integration with the underlying communication infrastructure. For instance, the messaging service 104 may receive inbound messages transmitted from the patient devices 102 over public communication networks, forward such messages to the system 100, and receive outbound communications from the system 100, delivering such communications to the patient devices 102. In alternative embodiments, the system 100 may incorporate the functionality of the messaging service 104, for example by directly interfacing with communication networks or telephony infrastructure to exchange communications with the patient devices 102 without an intermediate messaging service.

The system 100 includes a backend service 110, a database 120, and a user interface 130 accessible by healthcare providers. The backend service 110 serves as a central communication gateway for exchanging communications with external systems (such as the messaging service 104 or patient devices 102), e.g., via an external API, and for processing and routing inbound and outbound communications internally within the system 100. In various embodiments, the backend service 110 interfaces with the database 120 to store and retrieve communications and associated data, and with the user interface 130 to provide message data for presentation to healthcare providers and to receive provider input to generate or initiate outbound communications. Communications between the backend service 110, the database 120, and the user interface 130 may be facilitated via one or more internal APIs, network protocols, or other communication mechanisms.

In general, the system 100 may be implemented by a single computing device or by a network of computing devices. In various embodiments, the system 100 is implemented using a distributed computing architecture in which the backend service 110 and database 120 are hosted on one or more servers, such as cloud-based servers accessible via the Internet or other suitable communication networks. The user interface 130 may likewise be hosted on the server(s) (e.g., as a cloud-based frontend service) and accessible by provider devices, such as desktop or mobile computing devices used by healthcare providers, over a network. For example, in some embodiments, the user interface 130 is implemented as a web-based application accessible via a browser. Alternatively, at least a portion of the user interface 130 may be implemented as a dedicated application or thin client application executed locally on the provider devices.

Within the backend service 110, a communication routing component 112 is configured to receive inbound messages, initiate outbound messages and other communications, and facilitate routing of communications within the system. Upon receipt of an inbound message, the communication routing component 112 may process the message and store data associated with the message in the database 120. In some embodiments, the backend service 110 may replace a contact identifier associated with the inbound message (such as a phone number) with an abstract patient identifier before storing the message. The backend service 110 may subsequently use the abstract patient identifier to associate the stored message with a particular patient without exposing the contact identifier to the user interface 130. Conversely, when initiating an outbound communication, the backend service 110 may resolve the abstract patient identifier to a corresponding contact identifier to enable delivery of the outbound communication, e.g., via the messaging service 104.

The backend service 110 may further include a message analysis component 114 configured to analyze the content of inbound messages, for example, to determine one or more attributes associated with each message, such as urgency, category, or status. The backend service 110 may also include a message logic component 116 configured to leverage analysis of inbound messages, e.g., determined attributes, to generate or select content for outbound communications, initiate notifications to the user interface 130, and/or trigger additional workflows. Data associated with inbound messages and the determined attributes may be provided to the user interface 130 for presentation to a healthcare provider, e.g., as message listings or indicators corresponding to the inbound messages.

The database 120 (e.g., an Amazon Web Services DynamoDB or another managed, scalable NoSQL or relational data storage service) may store communications and associated data, including inbound messages, outbound messages (and optionally records of other outbound communications, such as phone calls), and mappings between abstract patient identifiers and corresponding contact identifiers. In some embodiments, such mappings may be maintained in one or more data structures, such as a key-value store or relational table, that associates each abstract patient identifier with one or more contact identifiers (e.g., a patient's phone number and/or email address). The communication routing component 112 may access the database 120 to store and/or retrieve patient-provider communications and may access the identifier mappings to perform identifier substitution or resolution during inbound and outbound communication processing. The user interface 130 may obtain inbound messages and associated data either by receiving such data from the backend service 110 or by retrieving such data from the database 120 under control of the backend service.

The user interface 130 may receive data representing inbound messages and associated attributes from the backend service 110 and may present such data to a healthcare provider. In various embodiments, the data presented via the user interface 130 may include communications aggregated across a plurality of patients and associated with one or more healthcare providers, thereby enabling the healthcare provider to access, review, and manage communications for multiple patients through a unified interface. To facilitate efficient monitoring and response, the user interface 130 may visually organize and prioritize messages, e.g., based on attributes determined for each message. Furthermore, the user interface 130 may receive input from healthcare providers for outbound communications to the patient. Such provider input may take the form, e.g., of reply messages or instructions to escalate communication to other modalities, such as telephone or video sessions. The user interface 130 transmits the provider input to the backend service 110 for processing, causing the communication routing component 112 to initiate corresponding outbound communications in the manner described above.

In some embodiments, access to the user interface 130 may be controlled via an authentication system configured to verify the identities of healthcare providers and other users of the system 100. The authentication system may include one or more authentication services that manage user credentials, login operations, and access permissions, and may be implemented, for example, using a third-party authentication platform in communication with the backend service 110, or an internally managed authentication mechanism. Upon successful authentication, a healthcare provider may be granted access to communications associated with one or more patients assigned to that provider. In various embodiments, associations between healthcare providers and patients may be maintained in the database 120, for example using abstract patient identifiers, so that the backend service 110 can selectively provide communication data to the user interface 130 based on the authenticated user's permissions. In this manner, the system 100 may restrict access to communications to authorized users while supporting organization and management of communications across multiple patients.

Although FIG. 1 illustrates a particular arrangement of components, various alternative configurations are possible. For example, one or more components of the backend service 110 and the messaging service 104 may be integrated, distributed across multiple computing systems, or implemented using shared infrastructure. Similarly, communications between components may occur via one or more direct or indirect communication paths, including API-based interactions, messaging queues, or other network-based communication mechanisms.

FIG. 2 is a flowchart illustrating an example backend-mediated workflow 200 for facilitating communication between a patient and a healthcare provider, in accordance with various embodiments, as may be performed by the backend service 110 of the system 100 of FIG. 1. As shown, the workflow 200 begins with receipt, at the backend service 110, of an inbound message 202 from a patient device (e.g., patient device 102), for example via a messaging service 104. Upon receipt of the inbound message 202, the backend service 110 replaces a contact identifier associated with the inbound message with an abstract patient identifier assigned to the patient (operation 204), and then stores the message 202 and/or associated message data in the database 120 in association with that abstract patient identifier (operation 206) for subsequent retrieval, analysis, and presentation via a user interface 130.

The contact identifier may vary depending on the communication modality used for the inbound message. For example, for a text message or voice message, the contact identifier may be the phone number associated with a mobile phone from which the text message was sent, whereas for an email message, the contact identifier may be the email address of the sender. In some circumstances, such as during enrollment of the patient in a messaging program, the message may be a web-form submission, and the contact identifier may be an email, phone number, or other contact information entered into a corresponding field on the form. Replacing the contact identifier with an abstract patient identifier serves to provide a unique internal reference to the patient within the system without exposing the contact identifier to a healthcare provider. The abstract patient identifier may be assigned to the patient, for instance, during enrollment. A mapping between the abstract patient identifier and at least one contact identifier may be stored, for instance, in the database 120. In some embodiments, only one contact identifier is stored for each patient. For example, the contact identifier may be a phone number of a mobile phone with text message capability, enabling both text exchange and phone calls with the patient using the same contact identifier. In other embodiments, multiple contact identifiers, such as an email address and a phone number, are recorded to facilitate communications via different channels and communication modalities, such as message exchange via email in addition to phone calls.

After storage, the backend service 110, for example using the message analysis component 114, may optionally analyze the content of the inbound message (operation 208) to evaluate processing criteria or determine one or more message attributes that may serve as inputs to subsequent operations for message handling, as described further below with reference to FIGS. 4 and 5. Data representing the inbound message, optionally along with determined message attributes or other analysis results, is then provided to the user interface 130 in association with the abstract patient identifier (operation 210). In the user interface 130, a healthcare provider assigned to the patient may view the inbound message and/or associated message data and provide input for an outbound communication responsive to the inbound message. As described in more detail with reference to FIG. 3, the provider input may, for instance, take the form of a reply message or a request to initiate another form of communication. If provider input for an outbound communication is received via the user interface 130 (decision operation 212), the backend service 110, upon receipt of the provider input and the associated abstract patient identifier, resolves the abstract patient identifier to retrieve a corresponding contact identifier of the patient, and then initiates the outbound communication 216 (operation 214). If multiple contact identifiers are stored for the patient, the contact identifier used for the output communication 216 may be selected at least in part based on the associated communication modality.

In a separate, parallel path, the backend service 110, for example using the message logic component 116, may determine whether an automated response is to be generated (decision operation 218). The determination may be based on one or more conditions, including analysis of the inbound message (operation 208), predefined messaging rules, scheduling criteria, or other logic implemented by the message logic component 116. When an automated response is to be generated, content for an outbound message is automatically selected or generated by the backend service 110 (operation 220), for example using stored templates, rules, or dynamically generated content. The communication routing component 112 of the backend service 110 then retrieves a contact identifier for the outbound message based on the abstract patient identifier, and sends the outbound message including the automatically generated content to the patient.

Accordingly, the backend service 110 may initiate one or more outbound communications 216 responsive to the inbound message 202 (operation 214), based on provider input received via the user interface 130 and/or content generated automatically by the backend service 110. In either case, initiation of the outbound communication 216 includes resolving the abstract patient identifier to determine a corresponding contact identifier associated with the patient, for example by accessing a mapping stored in the database 120. The backend service 110 then uses the resolved contact identifier to initiate transmission of one or more outbound communications via the messaging service 104, without exposing the contact identifier to the user interface 130. The outbound communication is transmitted to the patient device 102, for example, via the messaging service 104 or another communication channel. In this manner, the workflow 200 enables both provider-initiated and automatically generated communications to the patient through a backend-controlled, privacy-preserving architecture in which routing is performed using abstract patient identifiers.

FIG. 3 is a flowchart illustrating an example bifurcated workflow 300 for facilitating both asynchronous and synchronous communications between a patient and a healthcare provider, in accordance with various embodiments, as may be performed using the user interface 130 in conjunction with the backend service 110 of the system 100 of FIG. 1. As shown, the workflow 300 begins when the user interface 130 receives, from the backend service 110, data representing an inbound message—such as a notification of the inbound message or, in some embodiments, the message itself (collectively 302)—in association with an abstract patient identifier (operation 304). Upon receipt of the message or notification thereof, an indicator corresponding to the inbound message is displayed in the user interface 130 (operation 306), for example, in the form of a visual symbol, message excerpt, or brief message description, optionally alongside or including message-related data such as a time of receipt of the message, the abstract patient identifier, and/or a name or identifier of the healthcare provider(s) assigned to the patient. In various embodiments, indicators of multiple inbound messages are aggregated across patients and/or healthcare providers and displayed together in a dashboard view, for example, in a single list or in multiple panels corresponding to different message categories. In some embodiments, to preserve patient privacy while allowing multiple providers to access inbound messages via a shared view, the displayed indicators do not disclose any of the message contents.

Responsive to selection 308 of a given indicator by a healthcare provider assigned to the respective patient, the corresponding inbound message itself is displayed in the user interface 130 (operation 310). For this purpose, the user interface 130, if it has not previously received the message, may retrieve the message from the backend service 110 or the database 120. (For providers not assigned to the patient, access to the message may be blocked.) Alongside the message, the name or abstract patient identifier of the patient may be displayed to identify the patient, but without exposing a contact identifier. The healthcare provider may then provide input 312 for an outbound communication. The provider input 312 may, for example, include a reply to be delivered to the patient in an outbound message. The healthcare provider may input such a reply by typing free-form text into a text input field, selecting one among multiple reply options from a drop-down list, or selecting and customizing a reply template. Alternatively to providing a textual reply, the healthcare provider may record an audio reply for delivery to the patient. In some instances, instead of providing a reply to the inbound message, the healthcare provider may request that communication transition to a phone or video call or other synchronous communication modality, e.g., by clicking or otherwise selecting a corresponding user interface element.

Upon receipt of the provider input 312, a determination is made as to whether the provider input specifies escalation to a call (decision operation 314). If escalation to a call is not requested, the user interface 130 transmits the reply together with the associated abstract patient identifier to the backend service 110 (operation 316). The backend service 110 resolves the abstract patient identifier to determine a corresponding contact identifier associated with the patient for messaging (operation 318), such as an email address or phone number for text messaging, and sends an outbound message including the reply to the patient (operation 320), for example via the messaging service 104. The outbound message may then be stored in the database 120 in association with the abstract patient identifier (operation 322). If escalation to a call is requested (decision operation 314), the user interface 130 transmits a call request together with the associated abstract patient identifier to the backend service 110 (operation 324). The backend service 110 resolves the abstract patient identifier to determine a corresponding phone number or other contact identifier associated with the patient for calls (operation 326), and initiates a call to the patient using the resolved contact identifier (operation 328), for example via the messaging service 104 or another communication service. A record of the call may be stored in the database 120 in association with the abstract patient identifier (operation 330).

In this manner, the workflow 300 enables a healthcare provider to review inbound messages, provide responses, and selectively escalate communications, while the backend service 110 maintains control over routing and identifier resolution such that contact identifiers are not exposed to the user interface 130.

FIG. 4 is a flowchart illustrating an example workflow for analyzing inbound messages to generate user-interface indicators and alerts, in accordance with various embodiments, as may be performed by the backend service 110 of FIG. 1. The workflow 400 is triggered by the receipt of an inbound message 402 with the associated abstract patient identifier at the message analysis component 114 of the backend service 110. The message analysis component 114 may analyze the message content (operation 404), for instance, using keyword detection, rule-based processing, and/or processing by one or more machine-learning models (e.g., a large language model (LLM) or other model configured for natural-language processing). As output of the analysis, the message analysis component 114 may determine one or more message attributes 408 associated with the inbound message (operation 406), such as urgency, category (e.g., mental health, trauma, post-operative care, prenatal or maternal care, etc.), sentiment (e.g., an emotional tone or state reflected in the message content, such as anxiety, distress, frustration, or calmness), status (e.g., a condition or state inferred from the message content, such as a request for assistance, a report of symptoms, a general inquiry, or a confirmation or acknowledgment), or other characteristics of the message.

For example, in some embodiments, the analysis involves detecting, within the message content, one or more predefined keywords or phrases associated with particular message characteristics. The keywords or phrases may have associated weights or scores reflecting their relative significance in evaluating the message content, allowing the analysis component 114 to compute one or more metrics based on keyword/phrase occurrences and the associated weights. For example, an urgency metric may be computed as the sum, over a list of keywords indicative of urgency, of the number of occurrences of the keyword weighted by the associated scores. Similarly, to determine a message category, separate metrics may be computed for multiple categories based on weighted occurrences of respective category-specific keywords/phrases, and the message may be assigned to the category associated with the highest metric value. In other embodiments, the message content may be fed into a suitably trained machine-learning model. For example, a classification model may serve to classify the message according to message categories, status types, or sentiment classes, whereas a regression model may be configured to output a quantifier of urgency or of sentiment on a linear scale. Further, an LLM may be used as a general-purpose analysis tool to determine any one or more attributes, as specified in the prompt, based on the message content.

The attributes 408 or other outputs resulting from the analysis of the message contents may be used as inputs to subsequent processing operations, including generation of user-interface indicators and/or determination of escalation conditions. In one processing path, the backend service 110 generates indicator metadata based on the message attributes (operation 410). The indicator metadata 412 may define one or more properties for presentation of the indicator corresponding to the inbound message within the user interface 130, such as a position within a list or queue, an ordering relative to other messages, and/or visual properties including color, iconography, or other display characteristics. For example, the relative urgency of messages may be reflected in the ordering of indicators within a list, or in color-coded urgency classes (e.g., red, orange, and yellow indicating different degrees of urgency). Messages belonging to different categories may be displayed in different respective panels or user-interface portions, or visually distinguished by respective intuitive icons used for the indicators (e.g., a brain for mental health, a medical cross for post-surgery care, and a baby bottle for neonatal care). Status or sentiments may likewise be captured in the color or iconography of the indicator. Visual and positional encodings of multiple attributes may be combined in one representation. In this manner, presentation of inbound messages within the user interface 130 may reflect attributes determined through backend analysis, enabling healthcare providers to efficiently identify and prioritize messages.

In a separate, preceding or concurrent processing path, the backend service 110 may evaluate whether the message attributes indicate distress, urgency, or another condition warranting escalation (decision operation 414). When such a condition is detected, the backend service 110 may generate an alert (operation 416), which may be delivered to a healthcare provider, for example, via the user interface 130 or another notification mechanism (e.g., email or text to the provider). Alerts associated with inbound messages may, thus, prompt timely review or intervention.

In this manner, the workflow 400 enables automated analysis of inbound message content to derive actionable attributes that drive both presentation of messages within the user interface 130 and escalation of communications where appropriate.

FIG. 5 is a flowchart illustrating an example workflow for automatically generating outbound messages to patients, in accordance with various embodiments, as may be performed by the backend service 110 of FIG. 1. Automatic outbound message generation may be triggered, as discussed with reference to FIG. 2, by the message logic component 116 during processing of an inbound message in response to certain trigger conditions being met. Conditions triggering automatic responses may include, for instance, an urgency of the inbound message, as determined based on analysis of the message content, exceeding a specified threshold warranting an immediate response, and/or a determination that the nature of the inbound message allows for an adequate response without human input, e.g., to transmit general information. In some embodiments, the message logic component 116 is configured to generate automatic responses to inbound messages by default (e.g., in parallel with a workflow transmitting the inbound message to the user interface for review by a healthcare provider). Additionally to being prompted by inbound messages, automatic messages may also be triggered by certain defined schedule events. For example, upon enrollment of a patient in a message program, automatic messages may be generated and sent to the patient at specified intervals in accordance with a fixed schedule. The schedule may be tied to a reference date or time based on the defined event, such as the date of a traumatic event (e.g., a sexual or other assault), a medical examination, a surgical intervention, the birth of a child, etc. In some embodiments, outbound messages may also be triggered by a specified lapse of time since the most recent inbound or outbound message in the message history for a patient. For instance, if the most recent outbound message calls for a response from the patient, the system may follow up after a certain amount of time has passed without a response being received.

As shown in FIG. 5, in response to a trigger event 502 for the generation of an outbound message, relevant patient context data may be retrieved, e.g., by the message logic component 116, based on the abstract patient identifier 506 (operation 504). The retrieved patient context data 508 may include, for example, an enrollment status of the patient, prior communications, one or more attributes of an inbound message underlying the trigger event 502 as determined from content analysis, or an elapsed time since a specified patient-related event. Based at least in part on the patient context data 508, the message logic component 116 may then generate or select content for the outbound message (operation 510). In some embodiments, content is selected from among a repository of outbound message templates, completed and/or adjusted to the particular patient based on the patient context data 508. In other embodiments, content is generated by an LLM, using a prompt that includes at least a portion of the patient context data. Combinations of template-based selection and LLM-based generation of content are also possible. The selection or generation of content may be subject to message rules or schemas 512 derived from structured domain-specific input obtained from relevant patients, healthcare providers (e.g., physicians, psychologists, nurses, etc.), or other subject-matter experts (e.g., victim advocates, first responders, etc.). The selection or generation of message content may further involve applying one or more communication-style constraints 514. For example, one constraint may operate to avoid language that exceeds a threshold level of personalization, so as to reduce the likelihood that the outbound messages will be perceived as intrusive. In another example, the constraints serve to ensure that the outbound messages are limited in the amount or complexity of information they provide (e.g., per message or within a defined time period).

Once content for the outbound message has been determined, the message logic component 116 may create an outbound message request (operation 516) that includes the message content, the abstract patient identifier, and optionally timing information specifying when the message is to be sent. The communication routing component 112 then resolves the abstract patient identifier to determine a corresponding contact identifier associated with the patient (operation 518), for example using a mapping stored in the database 120, and sends the outbound message to the patient (operation 520), for example via the messaging service 104 or another communication channel. The outbound message may then be stored in the database 120 in association with the abstract patient identifier (operation 522), thereby maintaining a record of outbound communications within the system.

In this manner, the workflow 500 enables automated, context-sensitive generation and delivery of outbound communications while maintaining abstraction of contact identifiers and centralized control of communication routing within the backend service 110.

The described system 100 and workflows 200, 300, 400, 500 may be used to enable communications between patients and healthcare providers in a variety of contexts. In one example, the system provides secure and controlled outreach channels to support victims of sexual assault, domestic violence, or other crimes. In such applications, preserving confidentiality and preventing unauthorized disclosure of contact information are particularly important. Beneficially, the use of abstract patient identifiers in accordance with this disclosure enables communication between victims and healthcare providers without exposing underlying contact identifiers, while ensuring that all interactions are routed through monitored and auditable communication channels. In one concrete implementation, sexual assault victims may enroll in a 31-day text-messaging follow-up program to provide resources and assistance following the assault. Within this program, automatic messages may be scheduled based on the elapsed time since the completion of a forensic examination of the patient following the report of the assault, in anticipation of the physical, psychological, legal, or social support needs of the patient over time. The messages at various points in time may include information related to, for instance, medication adherence, sexually transmitted infection testing, pregnancy prevention, counseling services, victim advocacy resources, legal processes, and/or follow-up healthcare.

In additional examples, the system may be applied in a variety of programmatic and clinical contexts, including programs supporting pregnant or postnatal individuals, mental health applications, and post-surgical care. In such contexts, the system may facilitate ongoing communication between patients and healthcare providers through a combination of time-based messaging and provider interaction using the user interface 130. Messages may be generated in accordance with predefined schedules or program protocols, for example as periodic communications delivered over a multi-day or multi-week interval, and may be tailored to patient context, progression, or anticipated needs. Patients may also submit inbound messages describing symptoms, concerns, or other conditions, which may be processed by the backend service 110 and presented to healthcare providers using indicators that facilitate prioritization, categorization, or monitoring. The abstraction of patient identifiers enables these communications to be conducted in a consistent and controlled manner while maintaining privacy and ensuring that communications are routed through managed and auditable channels. This supports structured communication workflows across diverse use cases.

FIG. 6 is a block diagram of an example machine 600 as may be used for implementing any one or more of the various components (e.g., backend, user interface, database) of the system of FIG. 1 and the workflows of FIGS. 2-5. In general, the machine may be a personal computer (PC), a tablet PC, a set-top box (STB), a personal digital assistant (PDA), a mobile telephone, a smartphone, a web appliance, a network router, switch or bridge, a server computer, a database, conference room equipment, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), or other computer cluster configurations.

As depicted, the machine 600 (e.g., computer system) may include a hardware processor 602 (e.g., a central processing unit (CPU), a graphics processing unit (GPU), a hardware processor core, or any combination thereof), a main memory 604 and a static memory 606, some or all of which may communicate with each other via an interlink 608 (e.g., bus). The machine 600 may further include a display unit 610, an alphanumeric input device 612 (e.g., a keyboard), and a user interface (UI) navigation device 614 (e.g., a mouse). In an example, the display unit 610, input device 612 and UI navigation device 614 may be a touch screen display. The machine may additionally include a storage device 616 (e.g., drive unit), a signal generation device 618 (e.g., a speaker), a network interface device 620, and one or more sensors 622, such as a global positioning system (GPS) sensor, compass, accelerometer, or other sensor. The machine 600 may include an output controller 624, such as a serial (e.g., universal serial bus (USB), parallel, or other wired or wireless (e.g., infrared(IR), near field communication (NFC), etc.) connection to communicate or control one or more peripheral devices (e.g., a printer, card reader, etc.).

The storage device 616 may include a machine-readable medium 626 on which are stored one or more sets of data structures or instructions 628 (e.g., software) embodying or utilized by any one or more of the techniques or functions described herein. The instructions 628 may also reside, completely or at least partially, within the main memory 604, within static memory 606, or within the hardware processor 602 during execution thereof by the machine. In an example, one or any combination of the hardware processor 602, the main memory 604, the static memory 606, or the storage device 616 may constitute machine-readable media.

While the machine-readable medium is illustrated as a single medium, the term “machine-readable medium” may include a single medium or multiple media (e.g., a centralized or distributed database, and/or associated caches and servers) configured to store the one or more instructions 628. The term “machine-readable medium” may include any medium that is capable of storing, encoding, or carrying instructions for execution by the machine and that cause the machine to perform any one or more of the techniques of the present disclosure, or that is capable of storing, encoding or carrying data structures used by or associated with such instructions. Non-limiting machine-readable medium examples may include solid-state memories, and optical and magnetic media. Specific examples of machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; Random Access Memory (RAM); Solid State Drives (SSD); and CD-ROM and DVD-ROM disks. In some examples, machine-readable media may include non-transitory machine readable media. In some examples, machine-readable media may include machine-readable media that are not a transitory propagating signal. The instructions 628 may further be transmitted or received over a communications network 630 using a transmission medium via the network interface device 620. The machine 600 may communicate with one or more other machines utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, a Long Term Evolution (LTE) family of standards, a Universal Mobile Telecommunications System (UMTS) family of standards, peer-to-peer (P2P) networks, among others. In an example, the network interface device 620 may include one or more physical jacks (e.g., Ethernet, coaxial, or phone jacks) or one or more antennas to connect to the communications network 630. In an example, the network interface device 620 may include a plurality of antennas to wirelessly communicate using at least one of single-input multiple-output (SIMO), multiple-input multiple-output (MIMO), or multiple-input single-output (MISO) techniques. In some examples, the network interface device 620 may wirelessly communicate using Multiple User MIMO techniques.

Examples, as described herein, may include, or may operate on, logic or a number of components, modules, or mechanisms (all referred to hereinafter as “modules”). Modules are tangible entities (e.g., hardware) capable of performing specified operations and may be configured or arranged in a certain manner. In an example, circuits may be arranged (e.g., internally or with respect to external entities such as other circuits) in a specified manner as a module. In an example, the whole or part of one or more computer systems (e.g., a standalone, client or server computer system) or one or more hardware processors may be configured by firmware or software (e.g., instructions, an application portion, or an application) as a module that operates to perform specified operations. In an example, the software may reside on a machine-readable medium. In an example, the software, when executed by the underlying hardware of the module, causes the hardware to perform the specified operations.

Accordingly, the term “module” is understood to encompass a tangible entity, be that an entity that is physically constructed, specifically configured (e.g., hardwired), or temporarily (e.g., transitorily) configured (e.g., programmed) to operate in a specified manner or to perform part or all of any operation described herein. Considering examples in which modules are temporarily configured, each of the modules need not be instantiated at any one moment in time. For example, where the modules comprise a general-purpose hardware processor configured using software, the general-purpose hardware processor may be configured as respective different modules at different times. Software may accordingly configure a hardware processor, for example, to constitute a particular module at one instance of time and to constitute a different module at a different instance of time.

Although embodiments have been described with reference to specific examples, it will be appreciated that various modifications and changes may be made without departing from the scope of the invention. Accordingly, the specification and drawings are to be regarded in an illustrative rather than a restrictive sense.

What is claimed is:

1. A computer-implemented method comprising:
receiving, at a backend service, an inbound message from a patient;
replacing, by the backend service, a contact identifier associated with the inbound message with an abstract patient identifier assigned to the patient;
storing the inbound message in a database in association with the abstract patient identifier;
presenting, in a user interface, an indicator associated with the inbound message along with indicators associated with inbound messages from other patients;
upon selection of the indicator of the inbound message by a healthcare provider assigned to the patient, presenting the inbound message to the healthcare provider via the user interface;
receiving, from the healthcare provider via the user interface, provider input for an outbound communication to the patient;
transmitting the provider input in association with the abstract patient identifier to the backend service; and
initiating, by the backend service, the outbound communication to the patient based on the provider input, using a contact identifier associated with the patient for the outbound communication, the contact identifier being determined by resolving the abstract patient identifier,
wherein the contact identifier associated with the inbound message and the contact identifier associated with the patient for the outbound communication are not exposed to the healthcare provider via the user interface.

2. The method of claim 1, wherein the contact identifier associated with the inbound message and the contact identifier associated with the patient for the outbound communication each consist of a phone number associated with the patient.

3. The method of claim 1, wherein the provider input comprises a reply to the inbound message from the healthcare provider, and wherein initiating the outbound communication comprises sending the reply as an outbound message to the patient.

4. The method of claim 3, further comprising:
storing the outbound message in the database in association with the abstract patient identifier.

5. The method of claim 1, wherein the provider input comprises a request to place a call to the patient, and wherein initiating the outbound communication comprises placing the call, using a phone number associated with the patient.

6. The method of claim 1, further comprising:
automatically generating or selecting, by the backend service, with message logic that takes the abstract patient identifier as input, content for one or more outbound messages to the patient; and
sending the one or more outbound messages to the patient using a contact identifier associated with the patient for the outbound messages, the contact identifier being determined by resolving the abstract patient identifier.

7. The method of claim 1, further comprising:
determining, by the backend service based on analysis of content of the inbound message, one or more attributes associated with the inbound message, wherein the indicator presented in the user interface reflects the one or more attributes in at least one of placement or visual properties.

8. The method of claim 7, wherein the one or more attributes are determined by operating a trained machine-learning model on input comprising the content of the inbound message.

9. The method of claim 7, wherein the one or more attributes are determined based on detection of keywords in the content of the inbound message.

10. The method of claim 7, wherein the one or more attributes comprise an urgency associated with the inbound message, the method further comprising ordering the indicators based on relative urgencies.

11. The method of claim 7, wherein the one or more attributes comprise a category associated with the inbound message, the method further comprising presenting the indicator associated with the inbound message in a portion of the user interface associated with the category.

12. A computer system comprising:
a database configured to store inbound messages received from and outbound messages sent to patients, each in association with an abstract patient identifier assigned to the respective patient;
a backend service configured to:
upon receipt of an inbound message from a patient, replace a contact identifier associated with the inbound message with an abstract patient identifier assigned to the patient and cause the inbound message to be stored in the database in association with the abstract patient identifier; and
upon receipt of provider input for an outbound communication to a patient identified by an abstract patient identifier, initiate the outbound communication to the patient using a contact identifier associated with the patient for the outbound communication, the contact identifier being determined by resolving the abstract patient identifier; and
a user interface configured to:
present indicators associated with inbound messages, and upon selection of an indicator corresponding to one of the inbound messages by a healthcare provider assigned to a respective patient, present the inbound message to the healthcare provider without exposing the contact identifier associated with the inbound message; and
upon receipt of provider input for an outbound communication to a patient from a healthcare provider assigned to the patient, transmit the provider input in association with the respective abstract patient identifier to the backend service,
wherein contact identifiers associated with inbound messages and contact identifiers used for outbound communications are not exposed to the healthcare provider via the user interface.

13. The system of claim 12, wherein the backend service comprises message logic configured to, upon receipt of an inbound message, send a notification of the inbound message to the user interface, thereby initiating display, in the user interface, of an indicator associated with the inbound message.

14. The system of claim 12, wherein the user interface is configured to obtain inbound messages by at least one of:
receiving the inbound messages from the backend service; or
retrieving the inbound messages from the database.

15. The system of claim 12, wherein the backend service further comprises a message analysis component configured to analyze content of inbound messages from patients.

16. The system of claim 15, wherein the message analysis component is configured to analyze the content of the inbound messages using at least one of:
detection of keywords having associated weights; or
application of a machine-learning model.

17. The system of claim 15, wherein:
the backend service is further configured to determine, based on analysis of content of each inbound message, one or more attributes associated with the inbound message; and
the user interface is configured to present, for each inbound message, an indicator corresponding to the inbound message, the indicator reflecting the one or more attributes in at least one of placement or visual properties.

18. The system of claim 17, wherein:
the one or more attributes comprise at least one of urgency or category; and
the user interface is configured to at least one of order the indicators based on relative urgencies of the inbound messages or group the indicators based on categories associated with the inbound messages.

19. The system of claim 15, wherein the backend service further comprises message logic configured to automatically generate or select content for one or more outbound messages to the patient based on analysis of content of the inbound message or one or more attributes determined from the inbound message.

20. A non-transitory computer-readable medium storing instructions that, when executed by one or more processors of a server computer, cause the server computer to:
upon receipt of an inbound message from a patient, replace a contact identifier associated with the inbound message with an abstract patient identifier assigned to the patient;
cause the inbound message to be stored in a database in association with the abstract patient identifier;
cause an indicator corresponding to the inbound message to be displayed in a user interface;
receive, from the user interface, provider input for an outbound communication associated with the abstract patient identifier;
determine, based on the abstract patient identifier, a contact identifier associated with the patient for the outbound communication; and
initiate the outbound communication to the patient using the contact identifier associated with the patient for the outbound communication,
wherein the contact identifier associated with the inbound message and the contact identifier associated with the patient for the outbound communication are not exposed to the healthcare provider via the user interface.
